(19) United States
(12) Patent Application Publication
(10) Pub. No.: US 2026/0105662 A1
(43) Pub. Date: Apr. 16, 2026

(54) METHOD, DEVICE AND NON-TRANSITORY COMPUTER-READABLE MEDIUM FOR EDITING IMAGE

(71) Applicant: SHANGHAI UNITED IMAGING HEALTHCARE CO., LTD., Shanghai (CN)

(72) Inventors: Shang WU, Shanghai (CN); Xiang LIANG, Shanghai (CN); Chengcheng RONG, Shanghai (CN)

(73) Assignee: SHANGHAI UNITED IMAGING HEALTHCARE CO., LTD., Shanghai (CN)

(21) Appl. No.: 19/256,051

(22) Filed: Jun. 30, 2025

(30) Foreign Application Priority Data
Jun. 28, 2024 (CN) 202410865697.2

(51) Int. Cl. G06T 11/60; G16H 30/40

(52) U.S. Cl. CPC G06T 11/60; G16H 30/40; G06T 2200/24; G06T 2210/41

(57) ABSTRACT

The present disclosure may provide a method for editing image including: obtaining a first image from a server, the first image being a part of a sequence of medical images; and performing image editing on the first image by a user at a browser.

### CROSS-REFERENCE TO THE RELATED APPLICATIONS

This application claims priority of Chinese Patent Application No. 202410865697.2 filed on Jun. 28, 2024, the contents of which are hereby incorporated by reference.

### TECHNICAL FIELD

The present disclosure generally relates to the field of image processing, and more particularly, relates to a method, a device and a non-transitory computer-readable medium for editing image.

### BACKGROUND

In the field of image processing, there is a need to edit (e.g., outline, contour or segment) the image and further perform the operations based on the edited image.

As an example, typically, during radiotherapy, it is necessary to obtain contour data of the organ from the medical image of the patient. A subsequent radiotherapy plan is then developed for the radiotherapy based on the contour data.

### SUMMARY

According to one aspect of the present disclosure, a method for editing image is provided, the method including: obtaining a first image from a server, the first image being a part of a sequence of medical images; and performing image editing on the first image by a user at a browser.

In embodiments of the present disclosure, the method further includes: selecting the first image from the sequence of medical images at the server in response to instructions from the user before obtaining the first image from the server.

In embodiments of the present disclosure, the method further includes: obtaining the first image from the server after the image editing is determined to be performed based on the second image obtained from the server, wherein the first image is associated with the second image.

In embodiments of the present disclosure, the second image includes the predetermined contour, and the image editing method includes: determining that the contour editing is to be performed based on the predetermined contour; obtaining the first image from the server; and generating target contour data based on the contour editing of the first image by the user.

In embodiments of the present disclosure, the second image is obtained from the server.

In embodiments of the present disclosure, the second image includes a target area associated with the predetermined contour, and the first image includes the target area.

In embodiments of the present disclosure, determining that the contour editing is to be performed based on the predetermined contour includes: determining a contour to be edited based on the predetermined contouring contour; and determining that the contour editing is to be performed on the contour to be edited.

In embodiments of the present disclosure, determining a contour to be edited based on the predetermined contouring contour includes: determining the contour to be edited based on a selection of a corresponding predetermined contour in one or more predetermined contours by the user.

In embodiments of the present disclosure, after the contour editing is determined to be performed based on the predetermined contour in the second image, the method further includes: sending a first data request to the server, the first data request including an identifier corresponding to the contour to be edited; obtaining the third image sent by the server, the third image including a highlighted contour to be edited; determining a resulting contour to be edited based on the third image.

In embodiments of the present disclosure, determining that contour editing is to be performed on the contour to be edited includes: determining that contour editing is to be performed on the contour to be edited when a contour editing instruction from the user for the contour to be edited is detected.

In embodiments of the present disclosure, after the contour editing is determined to be performed on the contour to be edited, the method further includes: sending a second data request to the server, the second data request being used to request the server to send back first three-dimensional contour data corresponding to the contour to be edited.

In embodiments of the present disclosure, the image editing method further includes: converting first three-dimensional contour data to two-dimensional contour data based on a predetermined mapping relationship between a window coordinate system corresponding to a display window and a three-dimensional coordinate system corresponding to the target area, and displaying the two-dimensional contour data in the display window.

In embodiments of the present disclosure, generating target contour data based on the contour editing of the first image by the user includes: generating the target contour data based on the contour editing of the displayed two-dimensional contour data by the user.

In embodiments of the present disclosure, the above-mentioned image editing method is implemented when the instruction is executed by a processor.

According to one aspect of the present disclosure, a image editing method is provided, the method including: determining a part of a sequence of medical images as the first image at the server; and sending the first image to a browser by the server, the first image being used for a user to perform image editing at the browser.

In embodiments of the present disclosure, in response to that the image editing is determined to be performed at the browser based on a second image obtained from the server, the server determines the first image associated with the second image.

In embodiments of the present disclosure, the image editing method includes: sending the second image to a browser configured to determine that contour editing is to be performed; sending a first image associated with the second image; the first image being used by the user for contour editing to generate target contour data.

In embodiments of the present disclosure, the second image includes a target area associated with a predetermined contour, and the first image includes the target area.

In embodiments of the present disclosure, the above-mentioned image editing method is implemented when the instruction is executed by a processor.

According to one aspect of the present disclosure, a device for image editing is provided, the device including: a first acquisition processor configured for obtaining a first image from a server, the first image being a part of a sequence of medical images; and an editing processor configured for a user to perform image editing on the first image at a browser.

In embodiments of the present disclosure, after the image editing is determined to be performed based on a second image obtained from the server, the first acquisition processor is configured to obtain the first image from the server, wherein the first image is associated with the second image.

In embodiments of the present disclosure, the device for imaging editing further includes a first determination processor configured for determining that the contour editing is to be performed on a second image obtained from a server based on a predetermined contour, the second image including the predetermined contour and an associated target area, and the first acquisition processor is configured for obtaining the first image associated with the second image from the server, the first image including the target area, and the editing processor is configured to generate target contour data based on the contour editing of the first image by the user.

In embodiments of the present disclosure, the second image is obtained from the server.

In embodiments of the present disclosure, the second image includes a target area associated with the predetermined contour, and the first image includes the target area.

In embodiments of the present disclosure, determining that contour editing is to be performed on a second image obtained from a server based on the predetermined contour includes: determining the contour to be edited based on the predetermined contour in the second image; and determining that the contour editing is to be performed on the contour to be edited.

In embodiments of the present disclosure, after the contour editing is determined to be performed based on the predetermined contour in the second image, the method further includes: determining the contour to be edited based on a selection of a corresponding predetermined contour in one or more predetermined contours by the user.

In embodiments of the present disclosure, after the contour editing is determined to be performed based on the predetermined contour in the second image, the method further includes: sending a first data request to the server, the first data request includes an identifier corresponding to the contour to be edited; obtaining a second image sent by the server, the second image including a highlighted contour to be edited; determining a resulting contour to be edited based on the second image.

According to one aspect of the present disclosure, a method for data transmission performed by an electronic device is provided, the method including: determining a target compression strategy for the server to send data for transmission after a communication connection with the server has been established; sending target data to the server, the target data including the target compression strategy and a transmission request for request the server to send the data for transmission to an electronic device; and obtaining the data for transmission.

In embodiments of the present disclosure, the electronic device includes a browser, and the communication connection includes an established WebRTC point-to-point connection between the browser and the server.

In embodiments of the present disclosure, the data for transmission includes medical image data, and after the communication connection with the server is established, the method further includes: determining transmission parameters based on a bandwidth of the communication connection with the server, the transmission parameter being used to indicate a parameter requirement for each time the medical image data are sent by the server; and sending the transmission parameter to the server.

In embodiments of the present disclosure, determining transmission parameters based on a bandwidth of the communication connection with the server includes: according to the mapping relationship between the predetermined bandwidth range and one or more of the image resolution, frame rate, and bit rate, determining one or more of target resolution, target frame rate, and target bit rate for each time the medical image data are sent; and determining the one or more of the target resolution, the target frame rate, and the target bit rate as the transmission parameter.

In embodiments of the present disclosure, sending target data to the server includes: obtaining a first network quality at the current time; determining a first target transmission rate of the target data based on the first network quality; sending the target data to the server based on the first target transmission rate.

In embodiments of the present disclosure, the method further includes: exchanging data with the server using an end-to-end encryption method.

According to one aspect of the present disclosure, a method for data transmission which is applicable to a server is provided, the method including: obtaining target data sent by a electronic device after a communication connection with the electronic device has been established, the target data including a target compression strategy; compressing data for transmission corresponding to the target data according to the target compression strategy; and sending the data for transmission to the electronic device.

According to one aspect of the present disclosure, a device for data transmission is provided, the device being applied to a server and including: a compression strategy determination module configured to determine a target compression strategy for the server to send the data for transmission after a communication connection with the server has been established; a target data transmission module configured to send target data to the server, the target data including the target compression strategy and a transmission request for requesting the server to send the data for transmission to an electronic device; and a data acquisition module configured to obtain the data for transmission.

According to one aspect of the present disclosure, a device for data transmission is provided, the device being applied to an electronic device and including: a target data acquisition module configured to obtain target data sent by the electronic device after a communication connection with the electronic device has been established, the target data including a target compression strategy; a compression module configured to compress the data for transmission corresponding to the target data according to the target compression strategy; a data sending module configured to send the data for transmission to the electronic device.

In embodiments of the present disclosure, a non-transitory computer-readable medium on which an instruction is stored is provided, wherein the method for data transmission described above is implemented when the instruction is executed by the processor.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A is a flowchart of implementation of a image editing method according to an embodiment of the present disclosure;

FIG. 1B is a flowchart of implementation of a image editing method according to another embodiment of the present disclosure;

FIG. 1C is a flowchart of implementation of a image editing method according to yet another embodiment of the present disclosure;

FIG. 2 is a schematic illustration for an application scenario of a second image in a image editing method according to an embodiment of the present disclosure;

FIG. 3 is a schematic illustration of an implementation method for determining a contour to be edited in an image editing method according to an embodiment of the present disclosure;

FIG. 4 is a schematic illustration for an application scenario of a first image in a image editing method according to an embodiment of the present disclosure;

FIG. 5 is a flowchart of implementation of a image editing method according to another embodiment of the present disclosure;

FIG. 6 is a schematic illustration of an application scenario of two-dimensional contour data in a image editing method according to an embodiment of the present disclosure;

FIG. 7 is a schematic illustration of the structure of a device for image editing according to an embodiment of the present disclosure;

FIG. 8 is a schematic illustration of the structure of an electronic device according to an embodiment of the present disclosure;

FIG. 9A is a flowchart of implementation of a image editing method according to an embodiment of the present disclosure;

FIG. 9B is a flowchart of implementation of a image editing method according to another embodiment of the present disclosure;

FIG. 10 shows a flowchart of an interaction between an electronic device and a server in a image editing method according to an embodiment of the present disclosure;

FIG. 11 is a schematic illustration of the structure of a device for image editing according to another embodiment of the present disclosure;

FIG. 12 is a schematic illustration of the structure of a server according to an embodiment of the present disclosure;

FIG. 13 is a flowchart of implementation of a method for data transmission according to an embodiment of the present disclosure;

FIG. 14 is a schematic illustration of the structure of a device for data transmission according to an embodiment of the present disclosure;

FIG. 15 is a flowchart of implementation of a method for data transmission according to another embodiment of the present disclosure; and

FIG. 16 is a structural schematic illustration of a device for data transmission according to another embodiment of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

Following examples are used to describe the present invention, but they are only for illustrative objectives. Those skilled in the art may readily observe that various modifications and alterations of the device and method may be made without departing from the spirit and the scope of the invention. Hence, the claimed scope of the present disclosure should be based on the claims defined hereinafter.

Throughout specification and the claims, except for those further defined in the content, the meaning of the terms “a” and “the” may include the meaning of “one or at least one” element or component. Moreover, throughout the specification and claims, the singular terms may also refer to plural elements or components, unless the context clearly specifies that the plural usage is excluded. In the whole specification and claims, unless the content clearly specifies the meaning of some terms, the meaning of the term “wherein” includes the meaning of in/on something/somewhere”. The meaning of each term used in the present claims and specification refers to a usual meaning known to one skilled in the art unless the meaning is additionally annotated. Some terms used to describe the present invention will be discussed to guide practitioners to realize the present invention.

Further, various embodiments in the specification in are not meant to limit the claimed scope of the present invention.

The terms “substantially”, “around”, “about” and “approximately” can refer to a predetermined value, or a value difference within 20% or preferably within 10%. In addition, the quantities provided herein may be approximate, such that the terms “around,” “about” and “approximately” cab be used if the value or range is not further specified. When a quantity, density, or other values or parameters has a designated range, better range or listed ideal values, it should be viewed as including the range with any upper/lower limit or a same value, no matter the range is disclosed or not. For example, if it is described that the length of a component is X cm to Y cm, it is equivalent to the sentence “the length of the component is H cm, in which H may be any real number between X and Y.”

In the following description, specific details (such as the particular system structure, technology, etc.) are presented for illustrative purpose rather than limiting purposes in order to provide a thorough understanding of the embodiments of the present disclosure. However, it should be known to those skilled in the art that the present disclosure can also be implemented in other embodiments without those specific details. In other cases, detailed illustration of well-known systems, devices, circuits and methods are omitted so that unnecessary details will not obscure the description of the present disclosure.

It should be understood that when used in the description and the attached claims of the present disclosure, the term “includes”, “comprise” or “have” indicates the presence of the described feature, entity, step, operation, element and/or component, but does not exclude the presence or addition of one or more other features, entities, steps, operations, elements, components and/or their combination.

It should be noted that the information collection process (such as the face image collection process, fingerprint information collection process, etc.) and/or feature extraction process related to the present disclosure is carried out with the user's knowledge and permission. That is, the information collection process and/or feature extraction process complies with the requirements of laws and regulations and does not constitute an act that endangers public interests.

Additionally, in the description of the content specification and the attached claims, the terms “first”, “second”, “third”, etc. are only used to distinguish the description and should not be construed as indicating or suggesting relative importance.

When editing the image, the electronic device need to obtain a image, which is then edited (e.g., outlined) in a browser by a user, from a server through a browser-server (B/S) architecture. Specifically, for the sake of data security, organ contour data are usually stored in an internal server. As an example, when the browser retrieves all medical images and all contour data corresponding to the organ from the server, since there may be multiple contours to be outlined in one frame of medical image, and multiple frames of medical images are needed during radiotherapy. Therefore, the browser needs to obtain a large number of medical images and all the corresponding contour data from the server.

However, when the volume of medical images and contour data are large, the above approach is vulnerable to network communication delays. Also, when there are network fluctuations or insufficient bandwidth, the response speed may be slow, which dramatically affects the outlining operation that requires for real-time response.

The image editing method provided in the embodiments of the present disclosure may be applied to electronic devices. For example, the electronic device may include CT (Computed Tomography) device, MR (Magnetic Resonance) device, radiotherapy devices, tablet computer, notebook computer, ultra-mobile personal computers (UMPC), and netbooks, etc. The embodiments of the present disclosure do not impose any restrictions on the specific type of electronic device.

The present disclosure mainly provides examples of application of the image editing method in the medical field, but not limited to this, the image editing method can also be applied in other fields, e.g, the industrial field. As an example, with the image editing method referred to in the present disclosure, a contoured image may be obtained for generating plans for the radiation delivery, wherein the radiation delivery may include but is not limited to radiotherapy, radiation processing, etc.

It should be noted that the electronic device mentioned above may be a device installed with a browser for facilitating the contour editing by the user. This will enable the electronic device to interact with the server based on the networking function to complete the contour editing. In another embodiment, the electronic device can also be a device with a predetermined contour editing program installed. In this way, the electronic device can also implement the data interaction with the server to complete the contour editing while running the contour editing program.

However, if the electronic device has a predetermined contour editing program installed in it, the developed contour editing program will also need to be adapted to each of these operating systems due to the diversity of the electronic device's operating systems (for example, including various operating systems such as Windows, Linux, iOS, etc.). This further increases the difficulty of development, maintenance, and management.

Also, when the above contour editing program editing needs to be installed in the electronic device, a larger installation space is usually required. Therefore, for some electronic devices with poor performance or longer usage time, it will increase the workload of the electronic device and thereby reduce the efficiency of contour editing.

Based on this, an image editing method is provided in the present embodiment. The image editing method may be browser-based or browser-compatible and may be used on a wide range of internet-connected electronic devices (e.g., devices with a browser installed) without the need to develop additional contour editing programs adapted to multiple operating systems, reducing the difficulty of development, maintenance, and management. Moreover, since at least part of the data storage, processing and the like involved in the image editing method in this embodiment may be done with the server based on the network and the browser does not require a large installation space, it will not increase the workload of the electronic device, thereby ensuring the efficiency of contour editing.

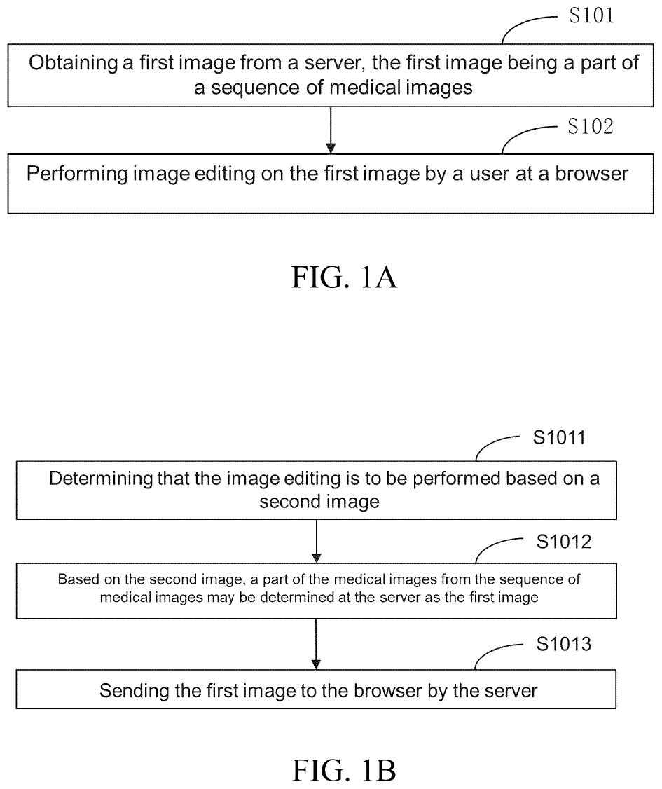

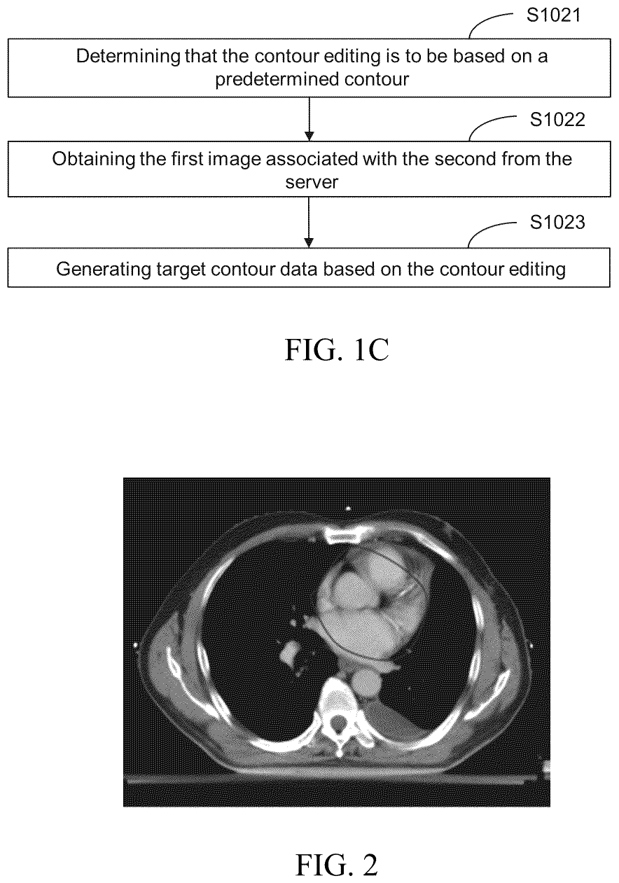

Referring to FIG. 1A, FIG. 1A shows a flowchart of the implementation of a image editing method according to the embodiments of the present disclosure. The method may be performed at a browser. The method may be performed by an electronic device. The electronic device may be configured to provide a browser.

The browser may be in communicative connection with the server, for example, via a wireless or wired network connection. The method could include steps S101 and S102.

At S101, the first image may be obtained from a server.

The first image may be part of a sequence of medical images (such as a CT image sequence or a MR Image sequence, which may include multiple medical images associated with each other).

Optionally, before the first image is obtained from the server, in response to the user's instructions, the first image may be selected from the sequence of medical images at the server. For example, after receiving the user's instruction to edit the image, the server can select a first image from the sequence of medical images and send it to the electronic device for displaying to the user at the browser.

Optionally, a second image may be provided to the electronic device providing the browser or other computing device, which is used for the user to determine or for the electronic device or computing device to automatically whether image editing is required. For example, the first image may be obtained from the server after it is determined that image editing is to be performed based on the second image obtained from the server. The first image may be associated with the second image.

For example, in an embodiment, the second image includes a sequence of images. After the user views the second image in a browser, an instruction to perform image editing for a particular image in the sequence is given by the user, and the instruction is then sent by an electronic device to a server for performing an operation to select a corresponding image, which corresponds to that particular image, from the sequence. The corresponding image is processed to obtain an editable or file-downsized second image which is then returned to the browser.

For example, in an embodiment, the second image includes one or more large-sized images with a predetermined contour. After the user views the second image in the browser, an instruction to perform image editing for the second image is given, which is then sent by an electronic device to a server, and the server performs operations to select a corresponding image, which corresponds to the second image, from the sequence of images acquired by the original scanning (i.e., the original images without a predetermined contour) and return the corresponding image directly to the browser.

In this disclosure, image editing may include, but is not limited to, one or more of contour drawing, contour modification, adjustment of window width and/or window position, cropping, stretching, color modification, contrast adjustment, compressing and the like for the image.

At S102, the image editing may be performed on the first image at the browser by the user.

The user can use various means such as mouse, keyboard, voice input and the like to determine whether the image editing needs to be performed on the first image. During the image editing by the user, the server and browser do not transfer the first image and/or data associated with the first image.

This ensures that the operation that requires a heavy load for data processing may be finished at the server rather than at the browser, while ensuring that the user can edit the image directly at the browser without having to communicate data with the server during editing, greatly reducing the burden of data communication and ensuring that image editing is not disturbed.

Referring to FIG. 1B, FIG. 1B shows a flowchart of implementation of an image editing method according to embodiments of the present disclosure. The method may be performed by an electronic device. The method may include steps S1011-S1013.

At S1011, the image editing may be determined to be performed based on a second image.

For example, the second image may be associated with the first image.

For example, the second image may be a sequence of images with predetermined contours or without predetermined contours, or a single image.

Image editing (e.g., contour editing) may be determined based on the second image either automatically by an electronic device or manually by the user via a browser.

For example, in an embodiment, the electronic device can automatically determine that no contour is outlined in the second image and thereby determine that contour editing is needed or required. In an embodiment, the user can view the second image in a browser and, upon finding that the second image has no contour or that the contour in the second image needs to be modified, manually confirm that the outlining needs to be performed by inputting a command or instruction.

At S1012, based on the second image, a part of the medical images from the sequence of medical images may be determined at the server as the first image.

For example, when the server receives instructions indicating the contour editing, one or more images in the sequence of medical images may be determined as the first image based on the second image. For example, the second image is an image with essentially the same content as the first image. As another example, the second image is an image with a predetermined contour, and the first image is the original image without a predetermined contour. Another example is that the second image is the original sequence of images originally captured, and the command indicates the contour editing for a specified image in said sequence, wherein the first image is the image corresponding to or the same as said specified image.

At S1013, the server may send the first image to the browser, the first image being used by the user to perform the image editing at the browser.

The first image may be sent from the server to the browser, where the user can perform image editing locally.

Referring to FIG. 1C, FIG. 1C shows a flowchart of implementation of an image editing method according to an embodiment of the present disclosure. The method may be performed by an electronic device. The method includes the following steps S1021 and S1022.

At S1021, the contour editing may be determined to be performed based on a predetermined contour, the second image including the predetermined contour. The second image may be obtained from the server. The second image may include the target area associated with the predetermined contour.

In an embodiment, the aforementioned server may be a device that stores contour data of the image and the target area, wherein the image may be the second image mentioned above, or any other image, as an non-limiting example, an image that does not include the predetermined contour. The predetermined contour may cover or enclose part or all of the target area, but not limited to this, the predetermined contour may also be associated with the target area in other ways. As non-limiting examples, the target area may be determined by the size of the field of view, by custom, by automatic division, etc. As non-limiting examples, the target area may be the lesion, the area near the lesion, or part or all of other human body parts. Also, as non-limiting examples, the above-mentioned target area may be an overall area of the human body or organ being scanned, or a part of the overall area. Predetermined contours may be either automatically outlined by a device or manually outlined by a person, and there is no limitation to the ways of acquiring the predetermined contour here.

In an embodiment, as non-limiting examples, the second image mentioned above may be a CT image, MR Image, infrared image, ultraviolet image, ultrasound image, etc. As non-limiting examples, the second image may be obtained by intercepting the contoured three-dimensional image, by intercepting the original three-dimensional image and then contouring it, by selecting from the contoured image sequence, by selecting from the original image sequence for contouring, or by taking a separate image and contouring it. The second image may contain the overall area of the human body captured, which may be pre-marked with one or more predetermined contours. A predetermined contour may correspond to a partial area of a human body part, or correspond to the entire area.

For example, referring to FIG. 2, FIG. 2 shows a schematic illustration of the application scenario of a second image in an image editing method according to an embodiment of the present disclosure. The second image in FIG. 2 includes only a predetermined contour (see the grey contour line in FIG. 2), which covers an area that may be regarded as part region of a human body. However, not limited to this, it can be contemplated that the second image could also include multiple predetermined contours.

In the present disclosure, the term “edit” may refer to, but not limited to, modifying, adjusting, moving, redrawing, subtracting, adjusting the color, adjusting the brightness, adjusting the line width, etc. The term “contour editing” may include, but not limited to, the editing operations for the contour's size, quantity and/or position such as modification, adjustment, movement, redrawing, deletion and the like, and may also include, but not limited to the editing operations for the contour's appearance such as color, brightness, line width and the like.

In an embodiment, the electronic device may first determine the contour to be edited in the second image based on the predetermined contour, and then determine that the contour editing is to be performed on the contour to be edited.

The contour to be edited may be one or more of the predetermined contours. For example, when only one predetermined contour is included in the second image, the electronic device can directly determine that the predetermined contour is the contour to be edited.

However, when the second image includes multiple predetermined contours, the electronic device may determine the contour to be edited based on the user's selection operation of the corresponding predetermined contour in one or more predetermined contours.

The selection operation includes, but not limited to, click, box selection, check, etc. It should be noted that when multiple corresponding predetermined contours are selected, all the selected corresponding predetermined contours may be regarded as the contours to be edited.

It should be noted that once the contour to be edited is determined, the electronic device may consider the contour to be edited as the contour that the user needs to reoutline. Thereby, the electronic device may enter the contour editing mode to determine the contour to be edited and perform the contour editing.

For example, when a user's contour editing instruction for the contour to be edited is detected, the electronic device can determine that the contour editing is to be performed on the contour to be edited. As an example, a user's contour editing instruction for the contour to be edited may be generated automatically or manually by the user. As non-limiting examples, for example, when the user selects one or more predetermined contours as the contour to be edited by mouse or touch, or when the user selects the contour to be edited by manually clicking the “edit” button on the user interface or by pressing a specific key, the contour editing instruction is initiated.

However, in the actual conditions, the contour lines of the predetermined contour included in the second image may not be clear, or the contour lines of multiple predetermined contours overlap, making it difficult for the user to intuitively understand the predetermined contour and the target area it covers. As a result, it is difficult for the user to determine whether they need to edit the selected contour to be edited.

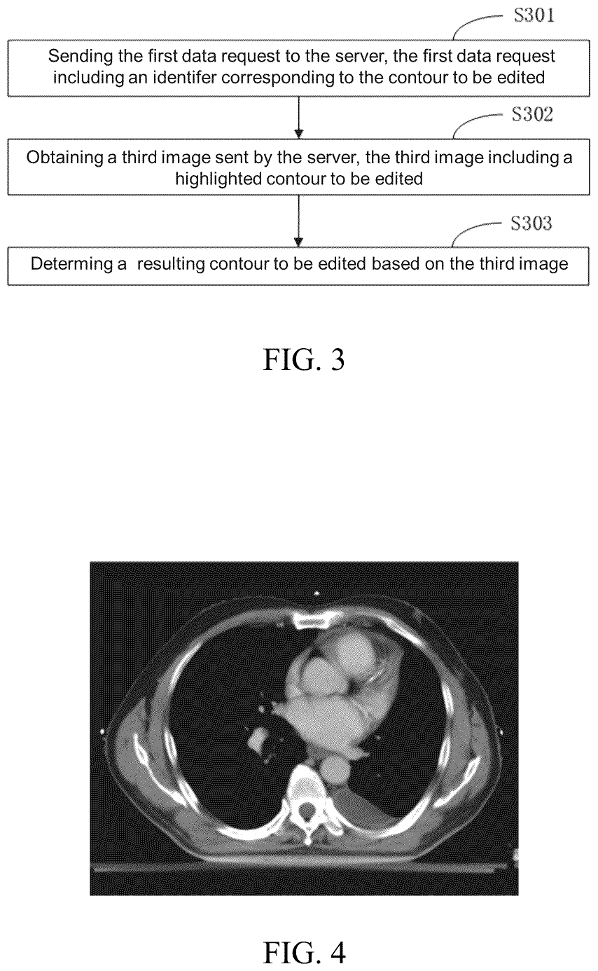

Based on this, in order to give users an intuitive understanding of the predetermined contour and the target area it covers to determine whether the contour to be edited needs to be edited, the electronic device can also perform steps S301-S303 as shown in FIG. 3 after the contour editing is determined to be performed based on the predetermined contour in the second image, so as to assist the user in determining the contour to be edited that actually needs contour editing. Details are described as follows.

At S301, the first data request may be sent to the server, the first data request including an identifier corresponding to the contour to be edited.

In an embodiment, as non-limiting examples, the above identifier may be a number, a letter, or a combination of both. It should be noted that the identifier should be unique so that the server can distinguish the selected contour to be edited.

It can be understood that the server, upon receiving the first data request, could respond to the first data request in order to send a third image to the electronic device.

At S302, the third image sent by the server may be obtained, the third image including a highlighted contour to be edited.

In an embodiment, it has been described above regarding to S301 that the server may distinguish the selected contour to be edited. Therefore, when sending the third image, the server may highlight the selected contour to be edited in the third image (e.g., highlight it with different color, with blinking display, with reverse color, with overlay shadow, etc.) to assist the user in observing the contour to be edited.

In an embodiment, when the selected contour to be edited is highlighted with different color, the server may adjust the color of said contour with a predetermined hue or brightness to make it highlighted in the browser of the electronic device. As non-limiting examples, the predetermined hue and/or brightness may be set according to the actual situations. It should be noted that the predetermined hue and/or brightness is at least greater than the hue and/or brightness of the predetermined contour that is not selected in the third image.

In another embodiment, when there are multiple contours to be edited, in addition to that the third image sent by the server may include the highlighted contours to be edited, one or more of the multiple contours to be edited may also be displayed in different colors relative to other ones. Furthermore, it helps the user to visually observe each contour to be edited as well as the associated target area, even when there is a large number of intersections among the contour lines of multiple contours to be edited.

At S303, the resulting contour to be edited may be determined based on the third image.

In an embodiment, since the contour to be edited that the user has selected is highlighted in the third image, it allows the user to carefully view the target area covered or enclosed by each contour to be edited. In this condition, the electronic device may again determine the resulting contour to be edited based on the user's selection or cancellation operation.

For example, when a user's cancellation operation on any contour is detected, it may be considered that this contour does not need to be edited.

Therefore, the cancelled contour to be edited may be redefined as the predetermined contour that is not selected. Also, when no user cancellation operation on the contour to be edited is detected, it may be considered that this contour to be edited is the resulting contour to be edited that truly requires contour editing as mentioned above.

Based on this, when a user's contour editing command or instruction for the contour to be edited is detected, the electronic device can determine the resulting contour to be edited as the contour that needs to be edited. Then, the electronic device may enter the contour editing mode.

At S1022, the first image associated with the second image may be obtained from the server. The first image may include the target area.

In an embodiment, the aforementioned server is the server that stores the first image. The number of servers may be one or more. In this condition, when the number of server is one, it may be considered that the server stores not only the second image but also the first image associated with the second image.

As non-limiting examples, the association between the second image and the first image may be set in advance in the server.

In an embodiment, the first image mentioned above may be an image that also includes the target area. However, the difference of the first image from the second image may be that the first image at this time may not include the contour line corresponding to the contour to be edited. For example, referring to FIG. 4, FIG. 4 is a schematic illustration of the application scenario of the first image in an image editing method according to an embodiment of the present disclosure. By comparing FIG. 4 with FIG. 2, it may be seen that the second image in FIG. 2 includes an image of the target area corresponding to the field of view covering the organ and its surrounding area, as well as a predetermined contour associated with the target area (which may be considered as the contour to be edited). However, the first image in FIG. 4 only includes the image of the organ corresponding to the target area, but does not include the contour to be edited associated with the target area. In this condition, the electronic device may subsequently edit the contour in the first image to generate the corresponding target contour data.

When performing contour editing in the first image, since the contour to be edited is not shown in the first image, it may enable the user not to confuse the contour line corresponding to the target contour data with the contour line of the contour to be edited during the contour editing process. This, in turn, enables user to outline the target contour data that better meets his/her needs.

In another embodiment, the first image mentioned above can also be a second image or a processed second image (such as a highlighted second image, an image obtained by extracting or intercepting a part of the target area from the second image, etc.) that contains the contour to be edited. The electronic device may subsequently perform the contouring operation directly in the second image or the processed second image to generate the corresponding target contour data. When performing the contouring operation in the second image or the processed second image, the user can use the original contour to be edited as a reference to quickly determine the contour that meets the requirements during contour editing, thereby improving the efficiency of generating target contour data. Alternatively, it can also improve the efficiency of generating the target contour data by allowing users to directly modify and adjust the original contour to be edited.

In another embodiment, when the first image is the processed second image as described above, the first image may also be an image containing the original predetermined contour or the contour to be edited. In this case, in the first image, each predetermined contour or contour to be edited may be displayed in one or more ways, as non-limiting examples, displayed with predetermined line type, predetermined display brightness, or predetermined color.

The predetermined line type may be a dashed line or a discontinuous line drawn with dots. As non-limiting examples, the above predetermined display brightness and predetermined color may be set according to the actual situation. However, it should be noted that in the first image, the predetermined line type, predetermined display brightness or predetermined color corresponding to each predetermined contour or contour to be edited should be different from the line type, display brightness and color of the contour corresponding to the target contour data, so that user can intuitively determine the contour corresponding to the target contour data. In an embodiment, there is no limitation on the predetermined line type, predetermined display brightness or predetermined color.

For example, when the line type is a dashed line, the contour line corresponding to the target contour data may be a solid line. Alternatively or additionally, when the predetermined display brightness is low brightness, the contour line corresponding to the target outline contour data may be high brightness. Alternatively or additionally, when the predetermined color is blue, the color of the contour line corresponding to the target contour data may be red.

Based on this, when users perform contour outlining operations in the first image, not only can they use the original contour to be edited as a reference, but also the target contour data generated by contour editing in the first image may be distinguished from the original predetermined contour or contour to be edited, without interference with the contour editing process.

Moreover, in another embodiment, when the first image is an image that does not include the contour line corresponding to each predetermined contour or the contour to be edited, the electronic device can also perform a split-screen operation on the display window, dividing the display window into a first window and a second window. At this point, the second image mentioned above may be displayed on the first window, and the first image may be displayed on the second window. Based on this, when users edit the contour of the first image in the second window, they may not only use the original contour to be edited as a reference, but also not be confused by the original predetermined contour or the contour to be edited. Consequently, there will be no interference with the contour editing process.

Based on the above description, it can be considered that the predetermined contour is not only used for the user to determine the contour to be edited in the second image, but also as a reference for the user to perform contour editing on the first image, so that the user can quickly determine the contour that meet the requirements when performing contour editing.

At S1023, a target contour data may be generated based on the contour editing of the first image performed by the user.

In an embodiment, based on the above explanation with respect to the first image in Step S102, it can be known that the electronic device may generate corresponding target contour data based on the contour editing of the first image performed by the user.

For example, in the target area corresponding to the contour to be edited, at least part of the area is of interest to the user. Therefore, it may be considered that the selected contour to be edited is an object that the user has the intention to edit. Based on this, it can be considered that the generated contour data for the target generally corresponds to the contour to be edited.

For example, the area enclosed or covered by the contour line corresponding to the target contour data in the first image may be at least partially the same as the target area corresponding to the contour to be edited. Alternatively, the area enclosed or covered by the contour line corresponding to the target contour data in the first image may completely contain the target area corresponding to the contour to be edited. Alternatively, the target area corresponding to the contour to be edited may be fully enclosed or covered by the contour line corresponding to the target contour data in the first image. There is no limitation to the above recitation.

In an embodiment, when the electronic device is a computer, the target contour data may be determined based on the running state of the mouse. For example, the electronic device may monitor the position and direction of the mouse's movement and, when a mouse click is detected, take the position coordinate of the position of the mouse click at that time as a coordinate point in the contour. Then, when the mouse clicks again, determine the corresponding coordinate point at that time and connect the previous coordinate point to the corresponding coordinate point at that time. Repeat the steps until the position coordinate of the last mouse click are the same as that of the first mouse click. Finally, various position coordinates in sequence are connected with each other to form the outline and determine it as the outline of the target. At this point, the position coordinate of the contour line corresponding to the target contour in the first image is the target contour data mentioned above.

During contour editing, the mouse click function can also perform one or more auxiliary editing functions such as deletion and modification, which will not be described in details.

In the field of radiation delivery, once the target contour data is determined, it may be used to determine the radiation delivery plan for the contoured area and/or the area near it, thereby ensuring precise and effective radiation delivery and avoiding excessive radiation doses.

It should be noted that the above contour editing is just one of the non-limiting examples. When the electronic device is another type of device, such as a tablet, mobile terminal and the like, the electronic device may generate target contour data by detecting the user's touch position. In an embodiment, there is no limitation on the way the user performs contour editing, or on the way the target contour data is generated.

In an embodiment, when a second image is obtained from the server, whether to perform contour editing and the corresponding target area when performing contour editing can be determined based on the predetermined contour associated with the target area in the second image. Then, when contour editing is performed, the first image associated with the second image may be acquired from the server again, at which point the first image may include only the target area. That is, there is no need to include the predetermined contour associated with the target area. As non-limiting examples, the first image may be obtained by intercepting a three-dimensional image, by outlining an image selected from the original sequence of images, or by taking a separate scan. Finally, target contour data may be generated based on the user's contour editing operation on the first image. Optionally, the electronic device can send the first image along with the target contour data to the server. In this case, the first image and the target contour data may be sent to the server as a combination, or the first image and the target contour data may be sent to the server independently of each other. There is no limitation here.

Based on this, using the above method, in the data interaction between the electronic device and the server, the data to be transmitted is only the first image and the second image that correspond to each other, rather than all the contour data corresponding to all predetermined contours (e.g., 3D contour data such as the closed curved surface surrounding the 3D volume of interest, the geometry, etc.) and the associated image data (e.g., 3D image data, such as 3D image covering the corresponding 3D volume of the target area). Moreover, during the generation of the target contour data, the electronic device does not need to obtain a large amount of contour data and/or contour process related data and communicate them with the server, thus reducing the amount of data communicated between the electronic device and the server. In this way, even if there is network fluctuation or insufficient bandwidth, the server's response speed may be improved due to the small amount of data that needs to be transmitted each time, reducing the impact of contouring operations that require high real-time communication performance.

In another embodiment, after obtaining the target contour data, the electronic device may also send the target contour data to the server for the server to convert the target contour data into a second three-dimensional contour data for storage.

In this case, a predetermined mapping relationship between the window coordinate system corresponding to the display window of the electronic device and the three-dimensional coordinate system corresponding to the target area may be pre-stored in the server for the conversion of the target contour data.

In an embodiment, the aforementioned window coordinate system may be set in advance according to the pixel size of the window display screen, and the three-dimensional coordinate system corresponding to the target area may be set by the staff during the acquisition of the second image, and the predetermined mapping relationship between the window coordinate system and the three-dimensional coordinate system may be determined, which will not be elaborated here.

It should be noted that based on the above explanations of the second image and the first image, it is known that when the user directly edits the contour of the first image, the contour line corresponding to the finally generated target contour data will be located on the first image.

However, as an example, in some cases, when the user performs the contour editing, only the position coordinates at the time of mouse click may be recorded, and the specific position coordinates of the contour line between the two position coordinates in the first image cannot be directly obtained. For example, when the contour between two position coordinates is an irregular arc, its exact position coordinates cannot be determined directly. As a result, the electronic device cannot send detailed target contour data to the server. As a result, there is a flaw when the server converts the target contour data to two-dimensional contour data.

Also, if the first image of the contour editing is sent directly to the server, not only will the amount of communicated data increase, but the server will also need to go through steps such as recognition, extraction, and transformation of the contour line corresponding to the target contour data in the first image in order to obtain a corresponding second three-dimensional contour data. Furthermore, it can also be known that there is a flaw when the target contour data are generated by the way of direct editing of the contour in the first image by the user.

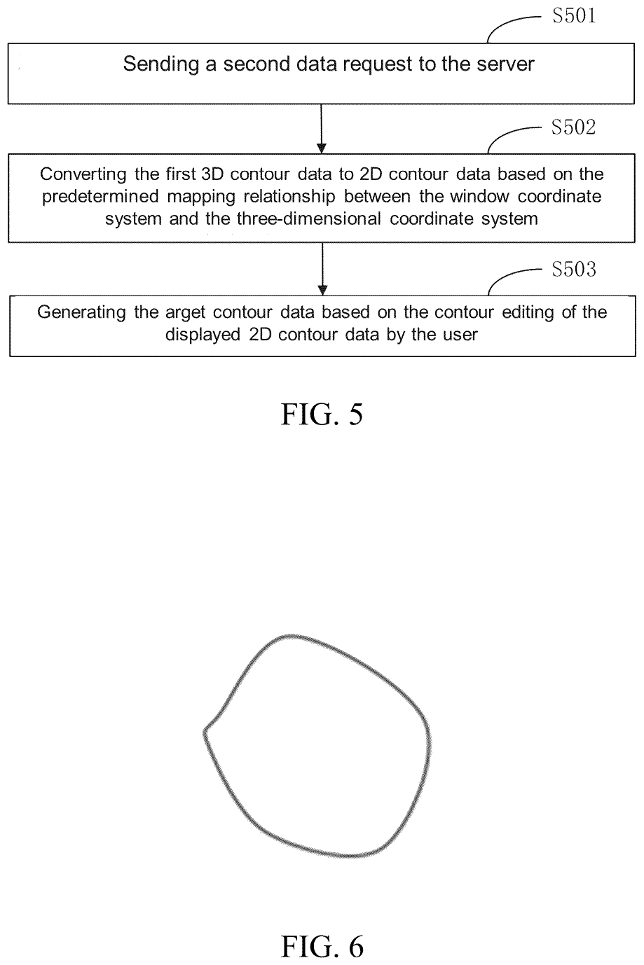

Based on this, in order to generate better target contour data, the electronic device may generate the target contour data according to Steps S501-S503 as shown in FIG. 5. The details are as follows:

At S501, a second data request is sent to the server, the second data request being used to request the server to send back the first 3D contour data corresponding to the contour to be edited.

In an embodiment, after the server receives the second data request, the first 3D contour data corresponding to the contour to be edited may be sent back to the electronic device.

It should be noted that the second data acquisition request may also contain a corresponding identifier of the contour to be edited for the server to perform a determination.

In another embodiment, after the electronic device has completed the above Steps S301-S303, the electronic device can send a second data request containing the identifier corresponding to the resulting contour to be edited to the server to obtain the first 3D contour data.

At S502, the first three-dimensional contour data is converted to two-dimensional contour data based on the predetermined mapping relationship between the window coordinate system corresponding to the display window and the three-dimensional coordinate system corresponding to the target area, and the two-dimensional contour data are displayed in the display window.

At S503, the target contour data are generated based on the contour editing of the displayed two-dimensional contour data by the user.

In an embodiment, the above predetermined mapping relationship has been explained above and will not be elaborated here. It should be noted that although the converted two-dimensional contour data may be displayed in the display window at this time, it will be independent of the first image. That is, although the converted two-dimensional contour data will be displayed in the display window at the same time and look like a part of the first image, the two-dimensional contour data will be displayed at a different layer from the first image.

For example, the electronic device can generate a transparent interface that is used to shield the first image and display the converted two-dimensional contour data on the transparent interface.

Based on this, when the user edits the contour of the displayed two-dimensional contour data, the generated target contour data may be located on the transparent interface so that the generated target contour data can be independent of the first image. Also, the interface may be transparent, so that the user can intuitively understand the display of the resulting target contour data in the first image when editing the contour data of the two-dimensional contouring, achieving the effect that what the user see is what the user get. That is, the display of the target contour data in the first image during the contour editing process is the same as the display of the target contour data in the first image after the contour editing is completed.

For example, referring to FIG. 6, FIG. 6 a schematic illustration of the application scenario of two-dimensional contour data in an image editing method according to an embodiment of the present disclosure. As can be seen from FIG. 6, the shape and/or size corresponding to the converted two-dimensional contour data is consistent with the shape and/or size of the predetermined contour (i.e., the selected contour to be edited) contained in the second image in FIG. 2.

In another embodiment, after performing the above Step S1021, after the contour editing of the second image obtained from the server is determined based on the predetermined contour, the electronic device may also send a second data request to the server first. Then, the server simultaneously sends the first image and the corresponding first 3D contour data to the electronic device. That is, the electronic device may perform the above Step S501 after the contour editing is determined to be performed on the contour to be edited when the contour editing command or instruction for the contour to be edited from the user is detected. At this point, the second data request will be used not only to request the server to send back the first 3D contour data corresponding to the contour to be edited to the electronic device, but also to request the server to send the first image to the electronic device. Ther is no limitation here.

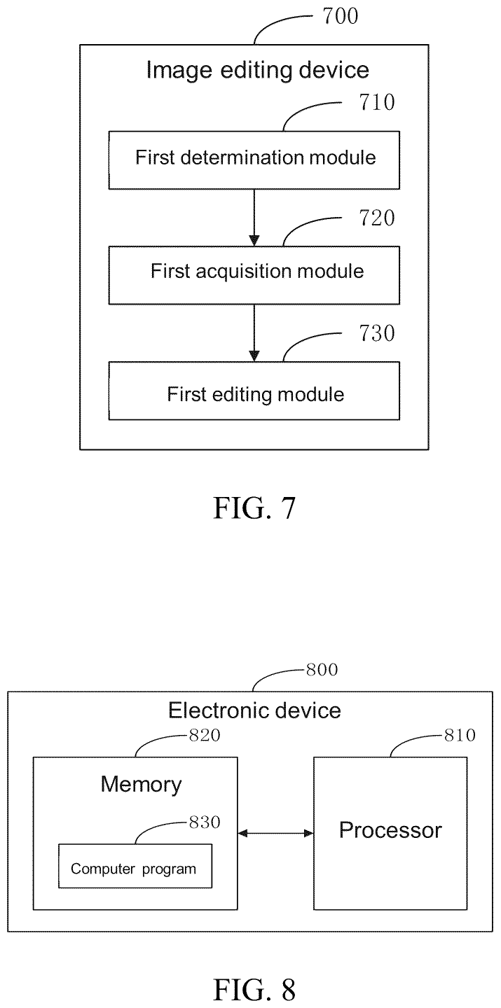

Referring to FIG. 7, FIG. 7 is a block diagram of an image editing device provided by the embodiments of the present disclosure. The modules included in the image editing device in the present embodiment are used to perform the steps in the embodiments corresponding to FIGS. 1A-1C, 3 and 5. Each module may be implemented as a processor. See FIGS. 1A-1C, 3 and 5 and the relevant description in the embodiments corresponding to FIGS. 1A-1C, 3 and 5 for details. For the sake of illustration, only the parts related to the present embodiment are shown. Referring to FIG. 7, the image editing device 700 may include a first acquisition module 720 and a editing module 730. Optionally, the image editing device 700 may also include a first determination module 710. These modules may be implemented as processors.

The first determination module 710, for example, may be configured to determine the contour editing of a second image obtained from the server based on the predetermined contour, the second image including the predetermined contour and the associated target area.

The first acquisition module 720 may be configured to obtain the first image from the server, the first image being a part of a sequence of medical images. After the image to be edited is determined based on the second image obtained from the server, the first acquisition module 720 may be configured to obtain the first image from the server. The first image may be associated with the second image. For example, the first acquisition module 720 may be configured to obtain the first image associated with the second image from the server, and the first image includes the target area.

The editing module 730 may be configured to the user to perform image editing on the first image at the browser. It is used to generate target contour data based on the user's contour editing operation on the first image.

In an embodiment, the first determination module 710 is also used for determining the contour to be edited in the second image based on the predetermined contour, and determining the contour to be edited for contour editing.

In an embodiment, the first determination module 710 is also used for determining the contour to be edited based on the user's selection operation on the corresponding predetermined contour in one or more predetermined contours.

In an embodiment, the image editing device 700 also includes a third sending module configured to send the first data request to the server, the first data request including an identifier corresponding to the contour to be edited.

The second acquisition module may be configured to obtain the second image sent by the server, the second image including a highlighted contour to be edited.

The second determination module may be configured to determine a resulting contour to be edited based on the second image.

In an embodiment, the first determination module 710 is also used for determining to perform contour editing on the contour to be edited when a contour editing command or instruction from the user for the contour to be edited is detected.

In an embodiment, the image editing device 700 also includes a fourth sending module configured to send a second data request to the server, and the second data request for requesting the server to send back the first 3D contour data corresponding to the contour to be edited.

The conversion module may be configured to convert the first 3D contour data to 2D contour data based on the predetermined mapping relationship between the window coordinate system corresponding to the display window and the 3D coordinate system corresponding to the target area, and display it in the display window.

The editing module 730 may be configured to generate the target contour data based on the contour editing of the displayed two-dimensional contour data by the user.

It should be understood that in the structural block diagram of the image editing device shown in FIG. 7, wherein each module may be used to perform each step in the embodiments corresponding to FIGS. 1A-1C, 3 and 5, and each step in the embodiments corresponding to FIGS. 1A-1C, 3 and 5 has been explained in details in the above embodiments. See FIGS. 1A-1C, 3 and 5 and the corresponding embodiments of FIGS. 1A-1C, 3 and 5 for more details, which will not be repeated here.

FIG. 8 is a block diagram of an electronic device according to an embodiment of the present disclosure. As shown in FIG. 8, the electronic device 800 of this embodiment includes a processor 810, a memory 820 and a computer program 830 that may be stored in the memory 820 and run on the processor 810, e.g., a program for an image editing method. When the processor 810 executes the computer program 830, it implements the steps in each embodiment of the above image editing methods, such as S101 to S102 as shown in FIG. 1A. Alternatively, when the processor 810 executes the computer program 830, the processor 810 may implement the function of each module in the embodiments corresponding to FIG. 7, for example, the function of each module shown in FIG. 7. The details can be seen in the relevant description in the embodiments corresponding to FIG. 7.

For example, the computer program 830 may be divided into one or more modules, wherein the one or more modules may be stored in memory 820 and executed by the processor 810 to implement the image editing method provided in the embodiments of the present disclosure. The one or more modules may be a series of computer program instruction segments capable of performing a specific function, and these instruction segment may be used to implement the execution procedure of the computer program 830 in the electronic device 800. For example, computer program 830 may implement the image editing method provided in the embodiments of the present disclosure.

The electronic device 800 may include, but not limited to, a processor 810 and a memory 820. It may be understood by those skilled in the art that FIG. 8 is merely an example of the electronic device 800 and does not constitute any limitations of the electronic device 800, which may include more or fewer components than shown, or a combination of certain components, or different components. For example, the electronic device may also include input/output devices, network access devices, buses, etc.

The processor 810 may be a central processing unit, or other general-purpose processor, digital signal processor, application-specific integrated circuit, field programmable gate array or other programmable logic device, discrete gate or transistor logic device, discrete hardware component, etc. A general-purpose processor may be a microprocessor, or this processor may be any common processor, etc.

In another embodiment, the above image editing method may also be applied to a server. The server may be a specific IT device that provides computing capability and runs software applications in a network environment, and the server can provide computing or application services for the aforementioned electronic device via the network. For example, as non-limiting examples, the server may provide services that respond to the first data request, the second data request, and send the second image, the first image, the second image, the third image and the first 3D contour data.

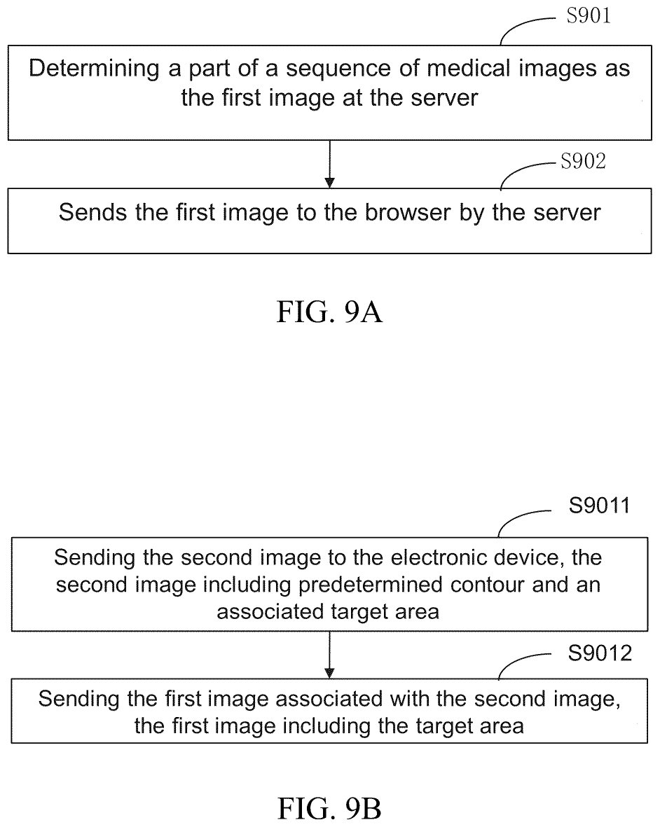

For example, referring to FIG. 9A, FIG. 9A shows a flowchart of the implementation of an image editing method according to an embodiment of the present disclosure, which includes the following steps.

At S901, a part of a sequence of medical images is determined from as the first image at the server.

At S902, the server sends the first image to the browser, the first image being used for the user to perform image editing at the browser.

Optionally, in response to the determination at browser to perform the image editing based on the second image obtained from the server, the server may determine the first image which is associated with the second image.

In an embodiment, details such as the first image and the image identifier have already been introduced in the above Steps S101-S102, which will not be elaborated here.

Specifically, referring to FIG. 9B, FIG. 9B shows a flowchart of the implementation of an image editing method according to an embodiment of the present disclosure, which includes the following steps.

At S9011, the second image is sent to the electronic device. The second image includes predetermined contour and an associated target area. The electronic device is used to determine, based on the predetermined contour, that the contour editing of the second image obtained from the server is to be performed.

At S9012, the first image associated with the second image is sent, the first image including the target area. The first image is used by the user to perform contour editing operations and generate the target contour data.

In an embodiment, the second image, the predetermined contour, the target area, the first image, and the target contour data have been explained in the above Steps S1021-S1023 and will not be elaborated further.

It should be noted that the server can not only perform the above Steps S901-S902, but also the above Steps S101-S102 and other steps in the embodiment of the Steps S501-S503. For example, the server may send the third image and the first 3D contour data corresponding to the contour to be edited to the electronic device and so on. More details can be known by referring to the examples in each of the above Steps S101-S102 and S501-S503, which will not be elaborated further.

It should be noted that in this embodiment, after the server sends the second image to the electronic device, the user can operate on the second image through the first electronic device to determine whether to perform contour editing and the corresponding target area when performing contour editing. Then, when performing contour editing, the server can again send the first image associated with the second image. At this point, the first image may include only the target area. That is, there is no need to include the predetermined contours associated with the target area, allowing the electronic device to generate target contour data based on the contour editing operations on the first image by the user. Based on this method, in the data communication between the electronic device and the server, only the corresponding second image and first image need to be transmitted, rather than the three-dimensional contour data corresponding to all the predetermined contours. In the process of generating the target contour data, the server does not need to send a large amount of contour data to reduce the amount of data communicating between the electronic device and the server. In this way, even if there is a network fluctuation or insufficient bandwidth, the server's response speed may be improved due to the small amount of data that needs to be transmitted each time, reducing the impact of contouring operations that require high real-time communication performance.

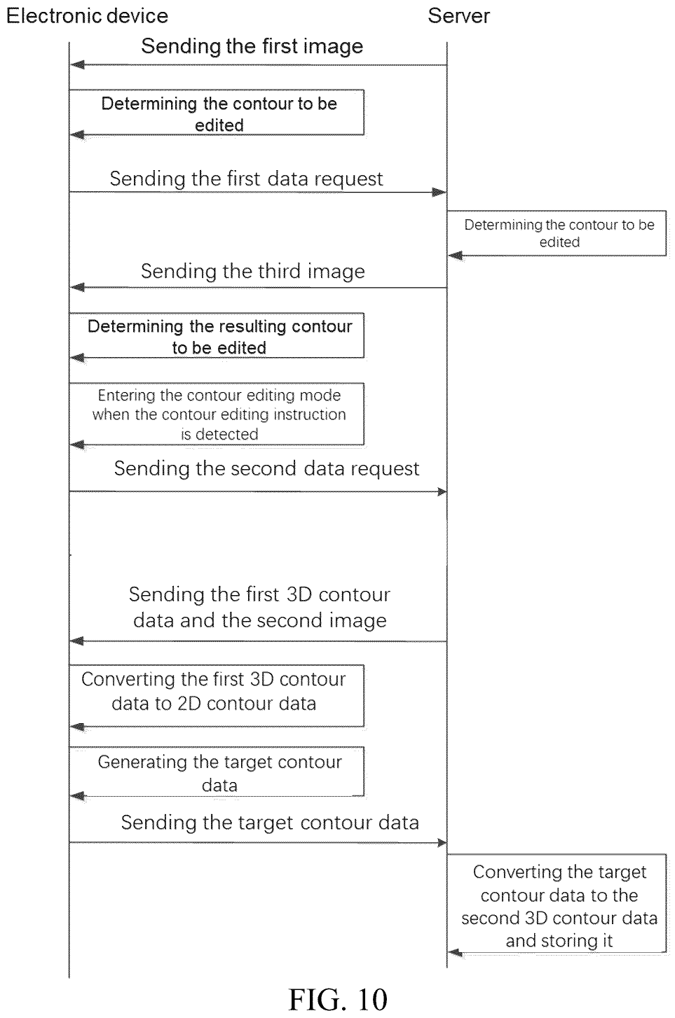

Referring to FIG. 10, FIG. 10 shows a flowchart of the interaction between the electronic device and the server in an image editing method according to an embodiment of the present disclosure. In this case, after the server sends a second image to the electronic device, the electronic device may determine the contour to be edited based on the user's selection operation in one or more predetermined contours. After that, the first data request containing the identifier of the contour to be edited is sent to the server.

The server may determine the contour to be edited based on the first data request and send a third image containing the highlighted contour to be edited to the electronic device to assist the user in determining the resulting contour to be edited.

The electronic device can display the third image and determine the resulting contour to be edited based on the user's cancellation operation. Then, when the user's contour editing command or instruction for the contour to be edited is detected, it is determined that the contour to be edited needs to be edited. At this point, the electronic device can enter the contour editing mode and send a second data request to the server.

After receiving the second data request, the server may send the first 3D contour data corresponding to the contour to be edited and the first image to the electronic device.

The electronic device may convert the first three-dimensional contour data to two-dimensional contour data based on the predetermined mapping relationship between the window coordinate system corresponding to the display window of the electronic device and the three-dimensional coordinate system corresponding to the target area, and then display it in the display window. Finally, based on the contour editing of the displayed two-dimensional contour data by the user, the target contour data is generated and sent to the server.

The server may reconvert the target contour data to the second 3D contour data for storage.

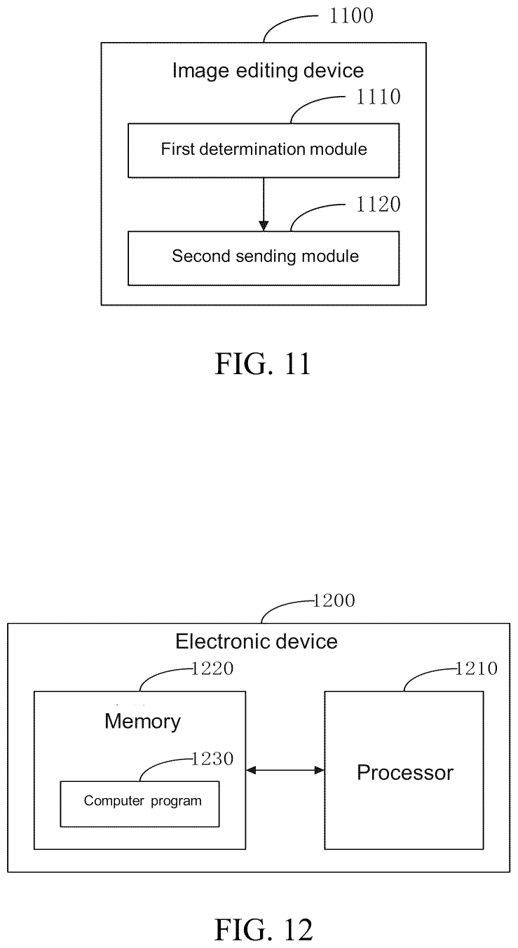

Referring to FIG. 11, FIG. 11 is a block diagram of an image editing device according to another embodiment of the present disclosure. The modules included in the image editing device in this embodiment may be used to perform the steps in the embodiments corresponding to FIGS. 9 and 10. More details can be known by referring to FIGS. 9 and 10, as well as the relevant descriptions in the embodiments corresponding to FIGS. 9 and 10. For the sake of illustration, only the parts related to the present embodiment are shown. Referring to FIG. 11, the image editing device 1100 may include the first sending module 1110 and the second sending module 1120.

The first sending module 1110 may be configured to send the second image to the electronic device. The second image may include predetermined contour and associated target areas. The electronic device may be used to determine the contour editing of the second image obtained from the server based on the predetermined contour.

The second sending module 1120 may be configured to send the first image associated with the second image, the first image including the target area. The first image may be used by the user to perform contour editing operations and generate target contour data.

It should be understood that in the structural block diagram of the image editing device shown in FIG. 11, the modules are used to perform the steps in the embodiments corresponding to FIGS. 9 and 10, and the steps in the embodiments corresponding to FIGS. 9 and 10 have been explained in detail in the above embodiments. More details can be known by referring to the relevant descriptions in FIGS. 9 and 10 and the embodiments corresponding to FIGS. 9 and 10. This will not be repeated here.

FIG. 12 is a block diagram of a server structure according to an embodiment of the present disclosure. As shown in FIG. 12, the server 1200 of this embodiment includes a processor 1210, a memory 1220 and a computer program 1230 stored in the memory 1220 and can be run on the processor 1210, such as a program for an image editing method. The processor 1210 implements the steps in each embodiment related to the above image editing method when the computer program 1230 is executed, such as S901 to S902 as shown in FIG. 9. Alternatively, when the processor 1210 executes the computer program 1230, the processor 1210 may implement the function of each module in the embodiments corresponding to FIG. 11, for example, the function of each module shown in FIG. 11, which can be known by referring to the relevant description in the embodiments corresponding to FIG. 11.

For example, the computer program 1230 may be divided into one or more modules, the one or more modules being stored in memory 1220 and executed by the processor 1210 to implement the image editing method provided in the embodiments of the present disclosure. The one or more modules may be a series of computer program instruction segments capable of performing a specific function, and these instruction segments may be used to describe the execution process of computer program 1230 in server 1200. For example, computer program 1230 may implement the image editing method provided in the embodiments of the present disclosure.

The server 1200 may include, but not limited to, processor 1210 and memory 1220. It may be understood by those skilled in the art that FIG. 12 is merely an example of the server 1200 and does not constitute a limitation of the server 1200, which may include more or fewer components than shown, or a combination of certain components, or different components. For example, the server may also include input/output devices, network access devices, buses, etc.

The processor 1210 may be a central processing unit, or other general-purpose processors, digital signal processors, application-specific integrated circuits, off-the-shelf programmable gate arrays or other programmable logic devices, discrete gates or transistor logic devices, discrete hardware components, etc. A general-purpose processor may be a microprocessor or the processor may be any common processor, etc.

The memory 1220 may be an internal storage unit of the server 1200, such as hard disk or memory of the server 1200. The memory 1220 may also be an external storage device for the server 1200, such as a plug-in hard drive, smart memory card, flash card which is prepared for the server 1200. Further, the memory 1220 may also include both the internal storage unit of the server 1200 and the external storage device.

The embodiments of the present disclosure provide a computer-readable storage medium on which instructions are stored. When the instructions are executed by a processor, an image editing method as described in each of the above embodiments is implemented.

The above examples illustrate that they are multiple embodiments of the image editing method for a user to perform data transmission between an electronic device and a server. However, it should be noted that, in addition to that the above approach can ensure that the server's response speed is not too slow and the impact on the contouring operations with high real-time requirements can be reduced even if network fluctuation or insufficient bandwidth exist, the mode of data transmission between electronic devices and servers can also be controlled in order to further ensure the stability of data transmission between the electronic device and the server. That is, the data transmission method described in the following multiple embodiments may be used to control the data transmission between the electronic device and the server, and increase the rate at which the server transmits data to the electronic device or obtains data from the electronic device (that is, increase the rate at which the electronic device obtains data from the server or sends data to the server). This, in turn, can further reduce the impact on the contouring operations that require high real-time communication performance.

The embodiments of the present disclosure provide a method, device and storage medium for image editing that can solve the problem of slow response speed of the server when contouring medical images obtained from the server in the existing technology. The technical benefits of the embodiments of the present disclosure compared includes that when a second image is obtained from the server, it is possible to determine whether to perform contour editing and determine the corresponding target area when performing contour editing based on the predetermined contours associated with the target area in the second image. Then, when contour editing is performed, the first image associated with the second image may be obtained from the server again, wherein the first image can include only the target area. That is, there is no need to include the predetermined contour associated with the target area. Finally, target contour data may be generated based on the user's contour editing operation on the first image. Based on this, using the above method, in the data communication between the electronic device and the server, only the corresponding second image and the first image need to be transmitted, rather than the three-dimensional contour data corresponding to all predetermined contours. In the process of generating the target contour data, the electronic device does not need to obtain a large amount of contour data and thus the amount of data transmitted between the electronic device and the server is reduced. In this way, even if there is a network fluctuation or insufficient bandwidth, the server's response speed may be improved due to the small amount of data that needs to be transmitted each time, reducing the impact on contouring operation that require high real-time communication performance.

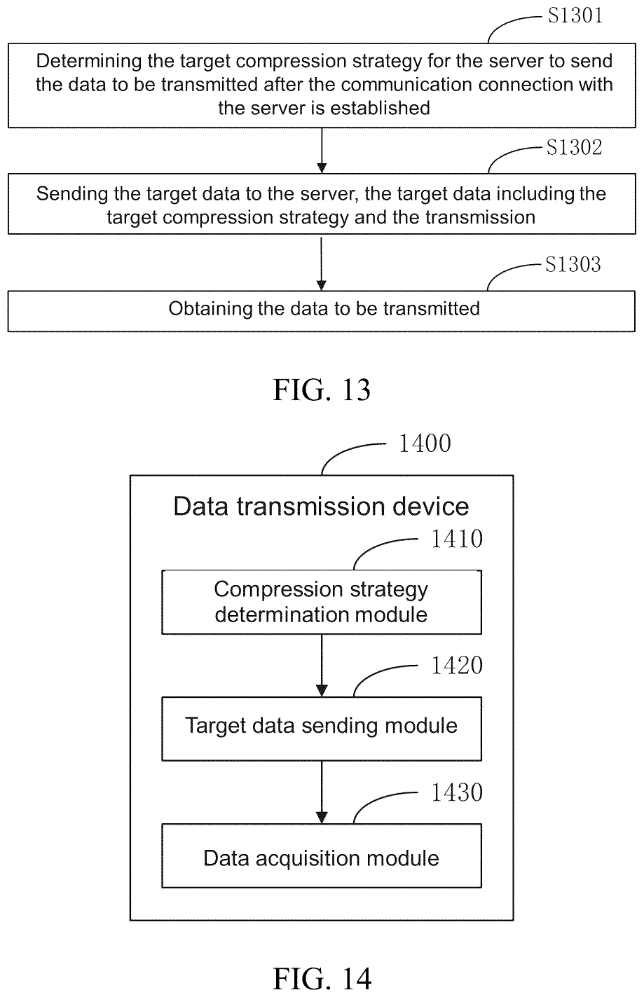

Referring to FIG. 13, FIG. 13 shows a flowchart of an implementation of a data transmission method provided by the embodiments of the present disclosure, which includes the following steps.

At S1301, the target compression strategy for the server to send the data to be transmitted may be determined after the communication connection with the server is established. The target compression strategy is used for the server to compress the data to be transmitted next time.

In an embodiment, the above-mentioned method may be applied to the electronic device in the embodiment corresponding to FIG. 1, and the server has been explained in the embodiment corresponding to FIG. 1 above and will not be elaborated here.

It can be understood that a communication connection with the server should be established before the data to be transmitted are obtained from the server. The data to be transmitted includes, but not limited to, image data such as the first image, the second image and the third image as described in the multiple embodiments above, and may also include non-image data such as the first three-dimensional contour data. There is no limitation to this. Also, the next data to be sent by the server may be determined by the server based on the received transmission request sent by the electronic device.

In an embodiment, as non-limiting examples, the means of establishing a communication connection include, but not limited to, HTTP (Hypertext Transfer Protocol) and TCP (Transmission Control Protocol), WebRTC (Web Real-Time Communication) and other methods.

For example, a WebRTC service may be deployed in the server and relevant parameters may be set. For example, parameters such as ports and encryption protocols may be set. In this way, the electronic device may establish a WebRTC point-to-point connection between the browser and the server based on the above parameters set by the server. This, in turn, enables that there is no need for communicating via intermediate node devices when the electronic device simply communicate data with the server, which reduces the delay of data transmission.

For example, the server may be set with multiple ports and the encryption protocol corresponding to each port, and the browser in the electronic device can directly transmit data to the server based on the port number corresponding to the port and encryption protocol, without the need for network nodes to forward. Among them, WebRTC provides a wealth of network optimization tools, such as adaptive bit rate control, anti-packet loss algorithms, etc., which can effectively ensure the stability of data transmission in weak network environments.

In an embodiment, the target compression strategy may be either a lossless compression strategy or a lossy compression strategy. The lossless compression strategy may include the strategies in which the compressed data to be transmitted is decompressed to obtain exactly the same data as before compression. The lossy compression strategy may include strategies in which the data obtained by decompressing the compressed data to be transmitted will be missing some information compared to the data to be transmitted before compression.

For example, lossless compression strategies include, but not limited to, strategies such as WinRAR and WinZip. Also, lossy compression strategies include, but not limited to, strategies such as mp3, divX, Xvid, jpeg, rm, rmvb, wma, wmv, etc. There is no limitation to this.

It is understandable that, due to the reduced amount of information in the data to be transmitted after lossy compression, the data quality of the data to be transmitted to electronic devices will decline, but the transmission rate of the data to be transmitted to electronic devices may be increased. Also, since the amount of information in the data to be transmitted remains the same after lossless compression, although the transmission rate is lower, the quality of the data to be transmitted to the electronic device may be guaranteed to be higher.

As an example, the electronic device may determine the target compression strategy from multiple predetermined compression strategies based on the user's selection operation.

In an embodiment, the aforementioned multiple predetermined strategies may include one or more strategies corresponding to the aforementioned lossless strategies, as well as one or more strategies corresponding to the aforementioned lossy strategies. As non-limiting examples, the selection operation includes, but not limited to, click, check, etc.

It can be understood that since the target compression strategy is determined based on the user's selection operation, it may be considered that the target compression strategy reflects the user's choice regarding the real-time nature or precision (data quality) of the transmitted data. Therefore, when the server compresses the data to be transmitted based on the target compression strategy and then sends it, it can meet the user's needs and improve the user's experience.

In another embodiment, the server itself can also determine the target compression strategy based on the quality of the second network at the current moment. For example, when the server detects that the second network quality at the current moment is the first predetermined quality (e.g., low quality), it may be considered that the network transmission quality is poor at this time. Therefore, a lossy compression strategy may be used as the target compression strategy. In this way, the real-time communication performance of the data to be transmitted may be guaranteed. Also, when the second network quality is the second predetermined quality (e.g., high quality), it may be considered that the network transmission quality is good at this time. Therefore, a lossless compression strategy may be used as the target compression strategy. Furthermore, the data quality of the data to be transmitted may be improved while ensuring the real-time nature of the data to be transmitted.

The determination of the second network quality includes, but not limited to, the server's determination based on one or more indicators such as the bandwidth between the server and the electronic device, the delay at the current moment, and the data transmission rate within the historical predetermined time period.

For example, delay is the time it takes for data to be transmitted from one end of the network to the other. Therefore, when the delay is greater than the predetermined delay, the second network quality may be determined to be the first predetermined quality. Otherwise, if the delay is less than or equal to the predetermined delay, the second network quality may be determined as the second predetermined quality. As non-limiting example, the delay may be set according to the actual situation. For example, the above delay may be 100 ms.

At S1302, the target data may be sent to the server, the target data including the target compression strategy and the transmission request used to request the server to send the data to be transmitted to the electronic device.

In an embodiment, the aforementioned target data includes the target compression strategy and the transmission request. The transmission request may be used to request the server to send the data to be transmitted, at which point the server can compress the data to be transmitted according to the target compression strategy.

Taking the target compression strategy as a lossy compression strategy as an example, when the transmitted data is medical image data (for example, the first image, the second image and the third image described in the above embodiments), lossy compression may be achieved by deleting certain color parts of the image edges in the medical image data.

It can be understood that in the medical field, the main information of medical image data lies in the central area of the image. Therefore, deleting certain color parts of the image edges in medical imaging data does not affect image quality a lot. Therefore, lossy compression may be achieved by using the above method.

The above transmission requests include, but not limited to, the first data request and the second data request described in the above embodiments.

In another embodiment, the target data mentioned above may include only the data that the electronic device needs to transmit to the server. For example, the target contour data obtained by the electronic device is obtained after contour editing is completed. At this point, the electronic device can send the target contour data as the aforementioned target data to the server.

In an embodiment, the electronic device can send the target data to the server at a predetermined transmission rate. As a non-limiting example, the predetermined transmission rate may be set according to the actual situation.

However, in actual data transmission, network congestion may occur when the electronic device sends the target data at a fixed predetermined transmission rate due to network fluctuations or insufficient bandwidth, resulting in the target data not being transmitted to the server.

Based on this, in order to reduce the degree of network congestion, the electronic device can first obtain the first network quality at the current moment. Then, based on the first network quality, the first target transmission rate of the target data is determined and the target data is sent to the server at the first target transmission rate.

In an embodiment, the way the electronic device determines the first network quality may be similar to the way the server determines the second network quality mentioned above, which will not be elaborated further.

As an example, the electronic device may determine the first predetermined transmission rate as the first target transmission rate when the first network quality is the first predetermined quality. Also, the second predetermined transmission rate is determined as the first target transmission rate when the first network quality is the second predetermined quality. The second predetermined quality may be higher than the first predetermined quality, and the second predetermined transmission rate may be higher than the first predetermined transmission rate.

It can be understood that when the first network quality is the first predetermined quality, it can be considered that the network transmission quality is poor. At this point, using a lower first predetermined transmission rate for sending the target data will reduce the transmission rate of the target data at this time, but it will reduce the degree of network congestion to facilitating increased rate for sending data later.

Also, when the first network quality is the second predetermined quality, it may be considered that the network transmission quality is good at this time. At this point, sending the target data at a higher second predetermined transmission rate not only increases the transmission rate of the target data, but also does not increase the degree of network congestion.

Based on this, the electronic device can flexibly adjust the transmission rate of the target data in real time according to the quality of the first network at the current moment to adapt to changes in the network environment. In this way, the transmission rate of the target data may be guaranteed as much as possible while reducing network congestion.

In another embodiment, the electronic device can also adjust the historical transmission rate of the previous moment in real time based on the first network quality, and determine the adjusted historical transmission rate as the first target transmission rate of the current moment.

For example, when the first network quality is the first predetermined quality, it may be considered that sending the target data at the historical transmission rate is prone to causing network congestion. Therefore, in order to reduce network congestion, the historical transmission rate is reduced by the third predetermined transmission rate to obtain the first target transmission rate.

Also, when the first network quality is the second predetermined quality, it may be considered that the network transmission quality is still high when sending the target data at the historical transmission rate while causing no network congestion. Therefore, in order to further increase the transmission rate, the historical transmission rate may be increased by a third predetermined transmission rate to obtain the first target transmission rate.

The third predetermined transmission rate may be set according to the actual situation, and there is no limitation to this.

In another embodiment, in order to ensure the security and privacy of the target data during transmission, the target data can also be encrypted for transmission. The encryption method may include, but not limited to, key hash encryption, asymmetric encryption, symmetric encryption, etc.

In order to further ensure that the target data is not decrypted and accessed by intermediate node devices during transmission, the data transmission between the electronic device and the server (including the electronic device sending the target data to the server and the server sending the data to be transmitted to the electronic device) may be encrypted end-to-end.

In end-to-end encryption, the data sender encrypts the data that needs to be sent, and the data can only be decrypted by the data receiver. The intermediate node devices through which the data is transmitted between the data sender and the data receiver cannot decrypt the data. This ensures that data security and privacy are not compromised by the unreliability of the intermediate node devices.

In the embodiments of the present disclosure, the data sender and the data receiver may include the electronic devices and servers described above, and the data to be sent may include but not limited to the target data and the data to be transmitted as described above. There is no limitation to this.

At S1303, the data to be transmitted may be obtained.

In an embodiment, the server can also encrypt the data to be transmitted using the above end-to-end encryption method when sending the data to be transmitted to ensure the security and privacy of the data to be transmitted.

In another embodiment, the rate at which the server sends the data to be transmitted may be the fourth predetermined transmission rate. However, similar to the scenario where the electronic device sends the target data as mentioned above, in actual data transmission, network congestion may occur when the server sends the data to be transmitted at a fixed predetermined transmission rate (e.g., the fourth predetermined transmission rate) due to network fluctuations or insufficient bandwidth, which prevents the target data from being transmitted to the server.

Based on this, the server can also determine the second target transmission rate according to the quality of the second network at the current moment. The method for determining the second target transmission rate may be similar to the method for determining the first target transmission rate mentioned above, which will not be elaborated here.

It can be understood that according to the quality of the second network at the current moment, the server may flexibly adjust the transmission rate of the data to be transmitted in real time to adapt to changes in the network environment.

It should be noted that the second target transmission rate of the server is usually related to the bandwidth allocated when the electronic device communicates with the server. The bandwidth may be the “maximum data rate” at which the data may pass from one end of the network to the other in a unit of time. That is, the bandwidth between the electronic device and the server will limit the rate at which the server sends transmitted data to the electronic device, or the rate at which the electronic device sends target data to the electronic device.

Therefore, when the server sends the data to be transmitted, it may also be considered that the transmission requirements of the data to be transmitted are usually associated with the bandwidth between the electronic device and the server.

For example, when the data to be transmitted is medical image data, the electronic device may determine the transmission parameters based on the bandwidth between the device and the server. The transmission parameters are used to represent the parameters required by the server each time it sends medical image data. Then, the transmission parameters are sent to the server to instruct the server to send the medical image data based on the transmission parameters.

In an embodiment, as non-limiting examples, the above transmission parameters include, but not limited to, one or more of the target resolution, frame rate, and bit rate at each time the medical image data is sent.

The target resolution refers to the amount of information stored in the medical image data, which represents the number of pixels stored per inch of the image. The frame rate mentioned above may be the frequency (rate) at which bitmap images, which is called frames, appear consecutively on a display. That is, when the data to be transmitted includes medical image data (for example, including the first image, the second image and the third image in the above embodiments, etc.), the frame rate may be considered as the transmission rate of the data to be transmitted mentioned above. Also, the above bit rate is the number of bits of data transmitted per unit time during data transmission.

As an example, the electronic device can determine one or more of the target resolution, target frame rate, or target bit rate for each medical image data transmission based on the mapping relationship between the predetermined bandwidth range and one or more of the image resolution, frame rate, or bit rate. The electronic device can correspond to one or more of the target resolution, target frame rate, and target bit rate as the above-mentioned transmission parameters.

For example, the electronic device can determine the target bandwidth range of the bandwidth, and then determine one or more of the image resolution, frame rate, and bit rate corresponding to the target bandwidth range as the above-mentioned transmission parameters.

Based on this, the electronic device can reasonably determine the target resolution, target frame rate and target bit rate when sending medical image data according to the bandwidth between it and the server, which can ensure the transmission quality when the medical image data of the media stream is sent to the electronic device for display.

As a non-limiting example, the mapping relationship may be set in advance in the electronic device.

It should be noted that the target resolution, frame rate, and bit rate are all requirements for the transmission of medical imaging data in the form of media streams. It can be understood that when the data to be transmitted is non-media stream medical image data such as the first 3D contour data, the server can determine the second target transmission rate based only on the above embodiments and send the data to be transmitted based on the second target transmission rate.

In this embodiment, after establishing a communication connection with the server, the electronic device can determine the target compression strategy when the server sends the data to be transmitted, and then send the target data containing the target compression strategy and the transmission request to the server in order to request the server to compress the data to be transmitted next time and reduce the amount of data to be transmitted. In this way, the response speed of the data to be transmitted may be improved. Furthermore, by using the above method, where the electronic device as the data requester determines the compression strategy for the data to be transmitted that needs to be obtained, rather than the server as the data sender determines the compression strategy for the data to be transmitted, the resulting data to be transmitted compressed by the target compression strategy can better meet the requirements of the electronic device. Furthermore, on the basis of obtaining the data to be transmitted, one can reasonably choose the real-time nature or precision (data quality) requirements that the data to be transmitted needs to meet, in order to improve the user experience of the electronic device.

Based on the above description, in another embodiment, referring to FIG. 14, the present disclosure also provides a block diagram of a data transmission device. The data transmission device includes modules for performing each step in the embodiment corresponding to FIG. 13. More details can be known by referring to the relevant description in the embodiment corresponding to FIG. 13 for details. For the sake of illustration, only the parts related to this embodiment are shown. Referring to FIG. 14, the data transmission device 1400 may include the compression strategy determination module 1410, the target data transmission module 1420 and the data acquisition module 1430.

The compression strategy determination module 1410 may be configured to determine the target compression strategy when the server sends the data to be transmitted after the communication connection between the server and the server is established. The target compression strategy may be used for the server to compress the data to be transmitted next time.

The target data sending module 1420 may be configured to send target data to the server. The target data includes the target compression strategy and the transmission request used to request the server to send the data to be transmitted to the electronic device.

The data acquisition module 1430 may be configured to obtain the data to be transmitted.

It should be understood that in the structural block diagram of the image editing device shown in FIG. 14, the modules are used to perform each step in the embodiment corresponding to FIG. 13, and each step in the embodiment corresponding to FIG. 13 has been explained in detail in the above embodiments.

More details can be known by referring to the relevant description in the embodiment corresponding to FIG. 13, which will not be repeated here.

In another embodiment, the present disclosure also provides an electronic device, which may also include a processor, a memory, and a computer program stored in the memory and can be run on the processor, such as a program for a data transmission method. The processor implements the steps such as S1301 to S1303 as shown in FIG. 13 in embodiments of the data transmission method as it executes the computer program. Alternatively, when the processor executes the computer program, it implements the function of each module in the embodiments corresponding to FIG. 14, for example, the function of each module shown in FIG. 14, which is described in the relevant description in the embodiment corresponding to FIG. 14.

In this case, the processor, memory, and computer program are similar to the processor, memory, and computer program corresponding to FIG. 8 as described above, except that in this case, the steps implemented by the processor when executing the computer program are those in each embodiment of the above data transmission method, not those in each embodiment of the image editing method, which will not be elaborated further.

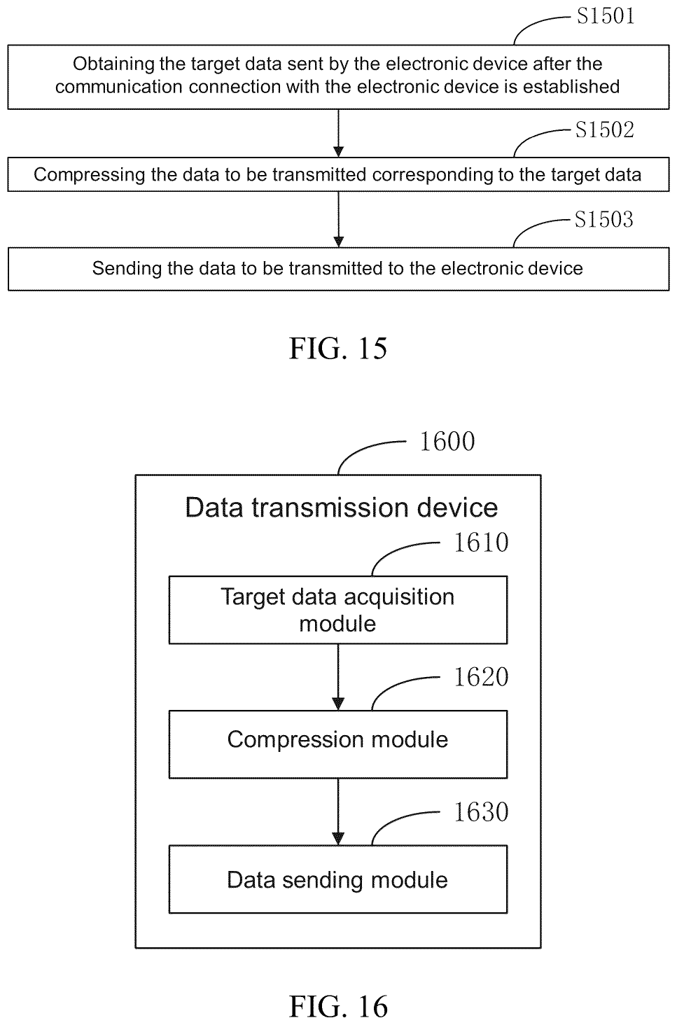

In another embodiment, the above data transmission method can also be applied to a server. The details of server has been explained in the embodiments of the above image editing method, which will not be further recited. Specifically, referring to FIG. 15, FIG. 15 shows a flowchart of implementation of a data transmission method according to the embodiments of the present disclosure, which includes the following steps.

At S1501, the target data sent by the electronic device may be obtained after the communication connection with the electronic device is established, the target data including the target compression strategy.

At S1502, the data to be transmitted corresponding to the target data may be compressed according to the target compression strategy.

At S1503, the data to be transmitted to the electronic device may be sent.

In an embodiment, the way the communication connection is established, the target data, the target compression strategy, the data to be transmitted, and the strategy for sending the data to be transmitted (e.g., the second transmission rate and transmission parameters described in the embodiments corresponding to FIG. 13 introduced above) have all been explained in the embodiment corresponding to FIG. 13 and will not be elaborated here.

It should be noted that when the server sends the data to be transmitted, if it detects packet loss during the transmission of the data to be transmitted, it can automatically retransmit to ensure the integrity of the data to be transmitted received by the electronic device.

The reasons for packet loss are usually various. For example, packet loss may be caused by network congestion, packet damage and denial of reception, or electronic device driver failure. There is no limitation to the reasons for packet loss.

As an example, due to the limited number of bytes a packet is able to carry, the data to be transmitted usually needs to be carried by multiple packets and sent to the electronic device. At this point, a header field in each packet will typically include an identifier field to store unique information about the packet. Among them, the first sent packet usually includes a start identifier and data information about the data to be transmitted (e.g., the number of packets, the size of the data to be transmitted, etc.), and the last sent packet usually includes an end identifier for the electronic device to determine that the data to be transmitted has been sent. After that, the electronic device can determine whether there is packet loss based on the identifier field and the data information. Furthermore, during the determination of packet loss, the target data packet corresponding to the target identifier field that needs to be retransmitted by the server may be determined based on the data information and the identifier field. Then, the retransmission request containing the target identifier field is sent to the server so that the server only needs to send the target data packet corresponding to the target identifier field. Then, while it is ensured that the electronic device can receive the complete data to be transmitted, the number of data packets that the server needs to resend may be reduced.

In this embodiment, after a communication connection with the electronic device has been established, the server may compress the data to be transmitted corresponding to the transmission request according to the target compression strategy sent by the data receiving end (the electronic device), and then send it back to the electronic device. Based on this, by using the above method, it may be considered that, after the data to be transmitted are compressed according to the target compression strategy determined by the electronic device as the data requesting end, the resulting data to be transmitted compressed by the target compression strategy can better meet the requirements of the electronic device. Furthermore, on the basis of obtaining the data to be transmitted, the electronic device can reasonably choose the real-time nature or precision (data quality) requirements that the data to be transmitted needs to meet according to its own needs, thereby improving the user experience of the electronic device.

Based on the above description, in another embodiment, referring to FIG. 16, the present disclosure also provides a block diagram of a data transmission device. The data transmission device includes modules for performing each step in the embodiments corresponding to FIG. 16. More details can be known by referring to the relevant description in the embodiment corresponding to FIG. 16. For the sake of illustration, only the parts related to this embodiment are shown. Referring to FIG. 16, the data transmission device 1600 may include the target data acquisition module 1610, the compression module 1620 and the data sending module 1630.

The target data acquisition module 1610 may be configured to obtain the target data sent by the electronic device after the communication connection between the electronic device is established. The target data includes the target compression strategy.

The compression module 1620 may be configured to compress the data to be transmitted corresponding to the target data according to the target compression strategy.

The data sending module 1630 may be configured to send the data to be transmitted to the electronic device.

It should be understood that in the structural block diagram of the image editing device shown in FIG. 16, the modules are used to perform each step in the embodiment corresponding to FIG. 15, and each step in the embodiment corresponding to FIG. 15 has been explained in details in the above embodiment. For details, please refer to the relevant description in the embodiment corresponding to FIG. 15, which will not be repeated here.

In another embodiment, the present disclosure also provides a server, which may also include a processor, a memory, and a computer program stored in the memory and can be run on the processor, such as a program for a data transmission method. The processor implements the steps, such as S1501 to S1503 as shown in FIG. 15, in each embodiment of the data transmission method as it executes the computer program. Alternatively, when the processor executes the computer program, it implements the function of each module in the embodiment corresponding to FIG. 16, for example, the function of each module shown in FIG. 16, which can be known by referring to the relevant description in the embodiment corresponding to FIG. 16.

In this case, the processor, memory, and computer program are similar to the processor, memory, and computer program corresponding to FIG. 12 above, except that in this case, the steps implemented by the processor when executing the computer program are those in each embodiment of the above data transmission method, not those in each embodiment of the image editing method, which will not be elaborated further.

The embodiments of the present disclosure provide a computer-readable medium on which instructions are stored, and when the instructions are executed by the processor, the data transmission method as described in each of the embodiments above is implemented.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “embodiments,” “an embodiment,” and/or “some embodiments” mean that a particular feature, structure or characteristic described in connection with the embodiment is included in at least embodiments of the present disclosure. Therefore, it is emphasized and should be appreciated that two or more references to “an embodiment” or “embodiments” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for editing image including:
obtaining a first image from a server, the first image being a part of a sequence of medical images; and
performing image editing on the first image by a user at a browser.

2. The method according to claim 1, further including:
selecting the first image from the sequence of medical images at the server in response to instructions from the user before obtaining the first image from the server.

3. The method according to claim 1, further including:
obtaining the first image from the server after that the image editing is determined to be performed based on the second image obtained from the server, wherein the first image is associated with the second image.

4. The method according to claim 1, wherein the second image includes a predetermined contour, and the method further includes:
determining that the contour editing is to be performed based on the predetermined contour;
obtaining the first image from the server; and
generating target contour data based on the contour editing of the first image by the user.

5. The method according to claim 4, wherein the second image includes a target area associated with the predetermined contour, and the first image includes the target area.

6. The method according to claim 4, determining that the contour editing is to be performed based on the predetermined contour includes:
determining a contour to be edited based on the predetermined contouring contour; and
determining that the contour editing is to be performed on the contour to be edited.

7. The method according to claim 6, determining a contour to be edited based on the predetermined contouring contour includes:
determining the contour to be edited based on a selection of a corresponding predetermined contour in one or more predetermined contours by the user.

8. The method according to claim 6, after the contour editing is determined to be performed based on the predetermined contour in the second image, the method further including:
sending a first data request to the server, the first data request including an identifier corresponding to the contour to be edited;
obtaining a third image sent by the server, the third image including a highlighted contour to be edited; and
determining a resulting contour to be edited based on the third image.

9. The method according to claim 6, determining that contour editing is to be performed on the contour to be edited includes:
determining that contour editing is to be performed on the contour to be edited when a contour editing instruction from the user for the contour to be edited is detected.

10. The method according to claim 6, after the contour editing is determined to be performed based on the predetermined contour in the second image, the method further includes:
sending a second data request to the server, the second data request being used to request the server to send back first three-dimensional contour data corresponding to the contour to be edited.

11. The method according to claim 10, further including:
converting the first three-dimensional contour data to two-dimensional contour data based on a predetermined mapping relationship between a window coordinate system corresponding to a display window and a three-dimensional coordinate system corresponding to the target area, and
displaying the two-dimensional contour data in the display window.

12. The method according to claim 11, generating target contour data based on the contour editing of the first image by the user includes:
generating the target contour data based on the contour editing of the displayed two-dimensional contour data by the user.

13. A non-transitory computer-readable medium with an instruction stored therein, when the instruction is executed by a processor, the processor perform the method according to claim 1.

14. A method for image editing method, including:
determining a part of a sequence of medical images as the first image at the server; and
sending the first image to a browser by the server, the first image being used for a user to perform image editing at the browser.

15. The method according to claim 14, wherein in response to that the image editing is determined to be performed at the browser based on a second image obtained from the server, the server determines the first image associated with the second image.

16. The method according to claim 14, including:
sending the second image to a browser configured to determine that contour editing is to be performed; and
sending a first image associated with the second image, the first image being used by the user for contour editing to generate target contour data.

17. The method according to claim 16, wherein the second image includes a target area associated with a predetermined contour, and the first image includes the target area.

18. A non-transitory computer-readable medium with an instruction stored therein, when the instruction is executed by a processor, the processor perform the method according to claim 14.

19. A device for image editing, including:
a first acquisition processor configured to obtain a first image from a server, the first image being a part of a sequence of medical images; and
an editing processor configured for a user to perform image editing on the first image at a browser.

20. The method according to claim 19, wherein the first acquisition processor is configured to obtain the first image from the server after the image editing is determined to be performed based on a second image obtained from the server, wherein the first image is associated with the second image.
